(19) United States
(12) Patent Application Publication
(10) Pub. No.: US 2013/0022686 A1
(43) Pub. Date: Jan. 24, 2013

(54) COMBINATIONS OF LIQUID FILTRATION MEDIA AND METHODS FOR ENHANCED FILTRATION OF SELECTED WATER CONTAMINANTS

(71) Applicants: Jerry E. Rademan, Jacksonville, FL (US); Robert H. Black, Jacksonville, FL (US)

(72) Inventors: Jerry E. Rademan, Jacksonville, FL (US); Robert H. Black, Jacksonville, FL (US)

(73) Assignee: HYDROPURE TECHNOLOGIES, INC., Jacksonville, FL (US)

(21) Appl. No.: 13/189,208

(22) Filed: Jul. 22, 2011

(51) Int. Cl. A61K 33/00; B01D 15/04; B01D 37/00; C02F 1/42; A61P 17/00; C02F 1/62; C02F 1/64; C02F 9/02; A61P 17/06; B01D 15/00; C02F 1/28

(52) U.S. Cl. 424/600; 210/252; 210/259; 210/205; 210/198.1; 210/660; 210/681; 210/663; 210/669; 210/679; 210/688; 210/682; 210/690

(57) ABSTRACT

By sequentially aligning various filtration media and delivery systems, enhanced synergistic reduction of water contaminants is obtained compared to the prior art or separate use of the individual media/filters. Specific filtration media are formulated with proper proportioning and sequencing to enhance the ability to reduce metals that cause staining, odors and bad taste such as iron, copper and manganese. Also disclosed is the reduction of potentially hazardous metal radionuclides metals such as uranium, iodine, cesium, plutonium and radium. Also disclosed is improved removal of heavy metals such as arsenic, lead, chromium, and mercury as well as organic compounds such as halogenated carcinogenic compounds. The present devices and methods remove specific bacteria and their toxins from water to reduce the risk of dermatitis. Thus, the present invention enhances our ability to achieving cleaner and safer water for drinking, swimming, washing, bathing and cooking.

### BACKGROUND OF THE INVENTION

1. Field of the Invention

The invention is in the field of liquid purification, such as water treatment. More particularly, this invention relates to filtration devices, systems and methods for use in water purification to remove metals and other contaminants.

2. Description of the Background Art

Hundreds, if not thousands of water filtration systems are known and/or are on the market that claim to remove or reduce various contaminants from either municipal or well-water sources. Most of these commercially available water filters use the same commonly known media such as activated carbon, sediment cartridge filters, ion exchange resins or others.

Metals found in well water or water running through rusty pipes are known to produce undesirable odors, bad taste and to cause staining. This is true for water used in swimming pools and spas as well as drinking water for homes, recreational vehicles, boats, industrial water treatments, and other related applications.

The common problematic metals include iron, copper and manganese. One example of this problem is encountered when filling or topping off water for a swimming pool or spa. Any forms of soluble or insoluble iron, copper or manganese in this fill water are instantly oxidize by the sanitizing chlorine, causing the metals to react with interior surfaces (e.g., plaster, vinyl, fiberglass) and leave unsightly discoloration. To remove these metals, some filter systems either selectively remove the metal ions through ion exchange, mechanically filter the metals, removal the iron with magnets; or oxidize and precipitate the metals for eventual removal. These all work with varying degrees of success, but are typically impractical due to the large amounts of treatment or filtration media required and the massive sizes of the filter devices.

The present invention provides novel solutions to preventing or minimizing these undesired effects.

Many heavy metals occur naturally, migrate into aquifers and find their way into drinking water supplies. Moreover, chemical spills and accidents or inadvertent drainage of heavy metals also contribute to contamination of municipal and well-water supplies. Known ion exchange resins, activated alumina, metal oxides and other media types have been used to remove or reduce each heavy metal, but each of the known systems and method have limited and varied degrees of success. A technology based on titanium dioxide called Metsorb® from Graver Technologies is described in U.S. Pat. No. 7,560,142 which discloses fibers containing a bound active material, such as a metal oxide of very small particle size that can be used for removal of metals and other contaminants from liquid solutions, e.g., arsenic, lead, mercury, uranium, etc. (See also U.S. Pat. No. 6,432,308 which discloses powdered nickel-based alloys to form tubular filter supports into the interiors of which are impregnated metal oxide particulates, preferably titanium oxide). Others such as U.S. Pat. Nos. 6,821,434 and 7,247,242 to Sandia National Laboratories describe the use of magnesium oxide and trivalent aluminum and divalent zinc-doped magnesium oxides to remove arsenic and other heavy metals. The present devices and methods go well beyond those noted above, even though the present invention may exploit some of the filtration media/methods disclosed in those documents.

A number of radionuclides occur naturally in the earth's soil and can get into water aquifers and end up in tap water. Processes employed in municipal water treatment plants may remove, at best, a small proportion of these hazardous materials which are potential carcinogens; well-water sources do not remove any of these contaminants. Additionally, industrial accidents including partial or total meltdowns of nuclear power plants release radionuclides that can travel great distances through air and water and gain access to water reservoirs and drinking water.

Though filter media that can remove or reduce some of these radionuclides are known in the art (“examples?), no single water filter system can remove them all. The present invention is addressed to devices and methods that remove all or most such radionuclides.

Water low in iron, e.g., from limestone deposits, has long been preferred for beer brewing and spirit drink distillation. Because of the contact with the limestone, this water was oxidizing to be free of iron and had pH>7, so that iron would be in the oxidized ferric state and deposited before use by a brewer or distiller. Such water also contributes calcium that is known to help control pH and improve yeast growth during fermentation. While originally unknown to brewers and distillers, one advantageous property of such water was its ability to control growth of certain microorganisms.

High iron favors bacteria that use iron, e.g., to make ribonucleotide reductase (RNR) enzymes. Low iron in the presence of manganese favors bacteria that utilize Mn. Oxidizing conditions in water serve to eliminate Clostridium perfringens bacteria. C. perfringens, responsible for disease like gas gangrene, produce the foul smell when water is allowed to remain stagnant and in the dark and are the third most common cause of food poisoning in the U.S. and United Kingdom. C. perfringens infections show evidence of tissue necrosis, bacteremia, and gas gangrene. The toxin involved in gas gangrene is known as α-toxin. Clostridium perfringens types A, B, C, D and E produce at least 12 different toxins that may be involved in pathogenesis, and have been named. α, β, ε and Λ toxin (‘major’ toxins), and δ, θ, κ (collagenase), λ (protease), μ (hyaluronidase), ν (deoxyribonuclease), γ and η toxin (‘minor’ toxins), and also an enterotoxin and neuraminidase (McDonel, J L, Pharmacology & Therapeutics 10: 617-655 (1980). In addition to producing such toxins, they produce foul odors if the water they contaminate is allowed to remain dark and stagnant. Disinfection of drinking water with chlorine to remove coliform bacteria does not, however, eliminate C. perfringens or their toxins.

The oxidizing conditions at water treatment plants allow C. perfringens to remain dormant only to be revived in the water distribution system in the presence of the organic materials that quickly make the water reducing. This is a particular problem in water from sources in lowland regions, for example, the Florida Keys and Miami, where the water never becomes sufficiently oxidizing enough to remove the organic material. Drinking such water containing C. perfringens toxins assaults the body and primes the immune system to react against additional antigens, including of these bacteria and their toxins. C. perfringens bacteria are not able to proliferate in the stomach and must compete with the common gut biota with which the human body exists in a somewhat symbiotic relationship. The C. perfringens bacteria are unable to produce significant quantities of toxin in the human intestines. Bathing, showering or washing with such water inoculates and thereby assault the skin, particularly in body regions of rough skin, causing dermatitis (skin inflammation) which leads to excess production of epidermis, amplifying skin roughness and causing the dermatitis to spread. Human skin contains little iron, and glands in the skin provide nourishment to microbes that contribute to maintenance of healthy skin at pH 5 and prevent colonization by other pathogenic microorganisms. The present invention is therefore beneficial in combatting the above skin problems.

### Products and Processes for Treating Water

Manganese (Mn) Greensand

The Mn Greensand process for the removal of Fe, Mn and H₂S from groundwater has been used in the U.S. since the 1950's. Two distinct treatment processes are associated with the use of Mn Greensand; specifically, the “IR” (intermittent regeneration) and “CR” (continuous regeneration) methods. Mn Greensand can, under certain conditions, remove Mn by catalytic oxidation.

Mn Greensand is processed from what is commonly known as New Jersey greensand but more precisely identified as glauconite, an iron, potassium, alumino-silicate material of marine origin. Glauconite occurs along the eastern coast of the U.S. where it was deposited approximately 75-80 million years. Greensand has been used since the 1920's originally as a natural zeolite for water softening due to its relatively high ion exchange capacity of approximately 3,000 grains/cu. ft. Until the development of higher capacity synthetic gel-type ion exchange resins, the greensand zeolites were an efficient and reliable part of the softening industry.

For Fe and Mn removal the naturally occurring singular nodular grains of glauconite are washed and classified to produce a filtration media with a pore size of 0.3-0.35 mm and a uniformity coefficient of 1.60 or less, conferring on the media excellent filtration characteristics. The glauconite is stabilized, then coated with Mn oxide in various valence states. This coating confers on the glauconite its special chemical oxidation-reduction properties for removal of Fe and Mn as well as small quantities of H₂S. A number of advantages of the Mn Greensand process over aeration and filtration are known, that is manifest as better reliability, flexibility, and a high quality effluent coupled with ease of operation. Fe and Mn removal by Mn Greensand in the CR process occurs by oxidation followed by physical removal of the resulting precipitates by filtration using a Mn Greensand or Mn Greensand-anthracite bed. In the IR method, the Mn is removed by contact oxidation. Generally, the CR method is used where Fe predominates with only small amounts of Mn, while the IR or catalytic oxidation process is used for water where Mn removal, with or without the presence of Fe, is required. Mn Greensand is an extremely heavy medium and has limited oxidation and metal removal capacities. Therefore, after metal removal capacities are exhausted, the Mn Greensand often needs to be regenerated by post-treatment with potassium permanganate to redeposit a MnO₂ surface onto the glauconite substrate. This is impractical for many applications where convenient, lightweight, fast and portable metal removal devices and procedures are required.

MetalTrap™ and PureStart™

The present inventors and colleagues developed several products for filtering and treating water that were designed to achieve some of the goals indicated above. The present invention represents further improvements in these products and completely new products that better achieve these goals.

MetalTrap™ (on the market since 2007) uses calcium carbonate, CaCO₃ (also abbreviated here as “CC”) as the first medium in the filter cartridge, followed by the immediate layering of MTM® (a trademark of Clack Corporation). MTM® (P/N MTM®) consists of a light weight granular core with a coating of MnO₂. The coating permits contact filtration where the media itself provides the oxidizing potential. This allows for a broader range of operation than many other iron removal media. Water at a pH level as low as 6.2 can be treated. Dissolved oxygen is not essential. MTM® reduces iron, copper, manganese, and hydrogen sulfide in water. Its active surface coating oxidizes and precipitates soluble Fe and Mn. Hydrogen sulfide is oxidized to a non-staining form of sulfur. The precipitates are filtered out in the granular bed and removed by backwashing. The ratio of CC to MTM® in MetalTrap™ ranges from 1:32 to 1:9.

Another product developed by some of the present inventors, PureStart™ consists of 100% activated carbon using Norit's HydroDarco® 4000 alone

Citation of the above documents is not intended as an admission that any of the foregoing is pertinent prior art. All statements as to the date or representation as to the contents of these documents is based on the information available to the applicant and does not constitute any admission as to the correctness of the dates or contents of these documents.

### SUMMARY OF THE INVENTION

The present invention provides systems, devices and methods for removing certain contaminants, primarily metals, from water, and more specifically from municipal water, from water feeding homes, swimming pools, spas, recreational vehicles, most preferably water being used for drinking, cooking, washing or bathing. This invention can also be used for industrial water treatment such as water cooling towers, water holding tanks and other related industrial applications. The approach utilizes a combination of filtration media, and steps, a critical one of which is comprises a manganese dioxide coated filtration medium.

More specifically, the present invention is directed to a liquid filtration system for removing or reducing the level of a contaminant from a liquid, preferably water. Other liquids that may be treated in accordance with this invention include oils, blood, juices, plasma, or molten metals for separation. The system comprises the following filtration devices or elements serially disposed:
(a) an optional mechanical pre-filter (not required when the levels of silt in the incoming water are adequately low);
(b) a bed of crystalline alkaline earth carbonate, preferably CaCO₃, MgCO₃ and/or Li₂CO₃, more preferably MgCO₃ and/or Li₂CO₃. The amount of this substance depends on the pH of the incoming water or the contaminant to be removed.
(c) an optional pre-oxidation chamber that releases calcium peroxide, magnesium peroxide or sodium percarbonate into the liquid and subsequent filter media;
(d) a bed of a MnO₂-coated medium, preferably zeolite or sand;
(e) a bed of granular and for powdered activated carbon optionally comprising silver ions at levels ranging from 0.001% to 3.0% (w/w); and
(f) an optional size exclusion or mechanical filter (to contain any activated carbon or MnO₂-coated medium fines.

Preferably the system is for removing contaminants that comprise an inorganic metal, such as Fe, Cu, Mn, Pb, As, Cr, or Co. The contaminant being removed may also be a radionuclide, preferably radioactive U, Cs, Pu, Ra, Co or I.

The contaminant being removed may also be a bacteria or a toxin produced by the bacteria.

The present invention also provides a method for removing or reducing the level of a contaminant from a liquid, preferably water, comprising:
(a) filtering the liquid through an alkali metal carbonate to obtain a first filtrate;
(b) treating the first filtrate with a filtration medium coated with a manganese dioxide. The preferred MnO₂-coated medium is MTM® or Manganese Greensand, described in more detail above and below.

The above may further comprise a step of filtering the liquid through a reducing or oxidizing ion exchange resin to synergistically react with contaminants that ion exchange resins alone are insufficient to bind and remove.

The above method may further comprise, before step (b), a step of
(c) treating the liquid with titanium dioxide.

The above method may further comprising after step (b) a step of:
(d) treating the liquid with granular or powdered activated carbon, which removes the above filtrate, any chlorinated hydrocarbons, metals and bioactive organisms. The activated carbon is preferably granular and may comprise 0.001 to 3.0% (w/w) silver ions that prevent or reduce bacterial growth in the liquid.

The above preferably comprises, prior to step (b), an optional step of treating the liquid with a powdered oxidizing agent which changes the valence state of inorganic metals to make it more filterable by subsequent filter media. Preferred oxidizing agents include calcium peroxide, magnesium peroxide or sodium percarbonate.

The above method may further comprising as a final step
(e) filtering the liquid through a filter having a pore size between about 0.1 and about 40 μm, preferably between about 0.1 and about 10 μm.

In the above method, the alkali metal carbonate is preferably CaCO₃, MgCO₃, Li₂CO₃ or a mixture thereof.

In the above method, the contaminant may be an inorganic metal, including a toxic heavy metal, including, but not limited to Fe, Cu, Mn, Pb, As, Zn, Co, Ni or Cr. In another embodiment, the contaminant is a radionuclide, preferably U, Cs, Pu, Ra, Co or I.

The contaminant may also be bacteria or other microorganisms, and a toxin produced by the bacteria. Preferred species of bacteria is Clostridium perfringens

The present invention includes a method of preventing or ameliorating a skin condition, disorder or disease in a subject having or being susceptible to the condition, disorder or disease, comprising providing to the subject a water supply used by the subject for bathing or washing water that has been treated by the above method or employing the above system. Preferably the condition, disease or disorder is dermatitis, eczema or psoriasis, most preferably dermatitis.

In a preferred embodiment of the above method, the water supply used by the subject is treated using following filtration and treatment steps:
(a) filtering the water through mechanical pre filter to removes silt if the silt level requires this, followed by
(b) filtering the water through crystalline CaCO₃, MgCO₃, Li₂CO₃ or a mixture thereof, if the pH of the incoming water is not sufficiently high, followed by;
(c) treating the water by controlled release of an oxidizing agent such as calcium peroxide, magnesium peroxide or sodium percarbonate, preferably calcium peroxide, which is preferably dispensed from a ceramic fiber bag with limited porosity to achieve such controlled release, followed by
(d) filtering the water through a MgO₂-coated medium such as sand or zeolite, preferably zeolite, followed by
(e) treating the water with granular or powdered activated carbon to which is preferably added a composition comprising 0.001 to 3.0% (w/w) silver ions; and followed by
(f) filtering the water liquid through a filter having a pore size between about 0.1 μm and about 40 μm, preferably 1 μm.
   Such treated, filtered water is then supplied to the subjects bathing or washing water supply.

In the above system or method, materials with sufficient porosity may be employed to hold small particle media to prevent bleeding into the other (subsequent) filtration media that could result in clogging and impeding fluid flow.

When using this system for use with a shower or sink to prevent or ameliorate a specific skin condition in a subject, the manganese coating on the medium may have a slow, controlled release rate. The controlled released manganese occurs from water flow erosion, and, with continued use, acts in itself as an anti-eczema or anti-psoriasis treatment.

### DESCRIPTION OF THE PREFERRED EMBODIMENTS

The unique contribution of the present invention is the sequencing and proportioning of some common and some uncommon filtration media to provide unexpectedly enhanced water filter performance for several related, albeit different, purposes. Provided herein are novel water filter systems that greatly reduce unwanted water contaminants by combining different chemical, biochemical and physical water treatment approaches not used before to achieve unexpectedly beneficial results.

The filter systems, devices and methods can be used at the Point of Entry (POE) (stationary filter systems) to treat water used for drinking, washing or bathing, in single family dwellings, multi-family dwellings, commercial buildings, government buildings, manufacturing and warehouse facilities, stadiums, etc.

The present filter systems, devices and methods can be used to treat water used for drinking, washing and bathing in any type of mobile vehicle, including recreational vehicles (RVs), motor homes, buses, airplanes, boats, trains, etc.

The present filter systems, devices and methods can be used at the Point of Use (POU) such as above or below sinks, as shower or bathtub filters, for institutional or residential beverage, washing machines, refrigerators, etc.

The present filter systems, devices and methods can be used for industrial water treatment systems such as cooling water towers, municipal and well-water treatment plants, home and industrial irrigation systems, water canals and waterways, etc.

The present filter systems, devices and methods can be used for any portable personal water filtration system.

The present filter systems and methods can be used for filling and refilling water “reservoirs” such as swimming pools, spas and water tanks.

A replaceable water filtration delivery system can employ use the present technology in the form of water filter tanks, filter housings with bags, canisters or cartridges of all sizes that utilize the filtration media described herein in the proper sequence and proportions. The above bags, canisters or cartridges used in the filter housings can be disposable and replaceable.

A non-replaceable filtration system can use the present technology in permanently fastened filters with hose connections, encased pipe systems, encased bags, bottles, etc.

### Removing or Reducing Metals in Waters

In one embodiment, the invention is directed to preventing or minimizing odors, bad taste and staining capacity of water used in various “containers” such as swimming pools and spas as well as for drinking water for homes, RVs, boats, and other such related applications

This method preferably employs calcium carbonate and/or magnesium carbonate as the first filtration medium with which incoming water reacts. This is immediately followed by a manganese dioxide coated filtration medium such as zeolite or sand, which may optionally be followed by a sediment filter to collect any bleeding of the oxidized or trapped metals from the other media.

An alkali earth metal carbonate, such as MgCO₃ or Li2CO₃ increases the pH sufficiently to convert the water-soluble metal ions to a water insoluble valence state (for example, ferrous to ferric or cuprous to cupric). Immediately after passing the metal-containing water through the carbonate, the metals are further oxidized by the MnO₂-coated medium to fully convert the metals to much larger water insoluble particles that are easier to filter physically from the water by the zeolite or other material that is coated with MnO₂.

Subsequently the water is preferably filtered physically by the use of a 0.1-10 μm sediment filter.

The ratio of alkali earth metal carbonate to MnO₂-coated media is preferably between 1:3 to 1:10, depending on the water pH, the metal content and temperature. This promotes an oxidizing filtration medium like MTM® or Mn Greensand to work much more effectively when compared to using the latter filtration media resins alone or in combination with other commonly used filter devices. It has been found that either MgCO₃ or Li₂CO₃, or combinations thereof, perform better than the previously used CaCO₃.

### Removing or Reducing Radionuclides in Water:

The present invention is directed to a device and method that properly sequences and proportions amounts of the appropriate filtration media to remove all or most of metal radionuclides of concern, including, but not limited to, uranium, iodine, cesium, plutonium and radium from a water source.

The preferred filtration system uses first a silicate sand medium to filter, for example, plutonium. This is immediately followed by the use of CaCO₃, MgCO₃ and/or Li2CO₃ filtration device/step, preferably CaCO₃ and/or Li₂CO₃, followed by MTM® or Mn Greensand to oxidize each of radionuclide being targeted to its highest valence state for easier removal, followed by an ion exchange resins based on either strong or weak acid cation or anion resins, either alone or in combination for removing or reducing cesium, followed by an activated carbon media for removing or reducing iodine, followed by the use of TiO₂ and/or magnesium oxide for removing or reducing uranium. Such a multi-tiered and sequenced system is used when removal of all the mentioned radionuclides are required. If only one or some of the radionuclides are to be reduced or removed, the use of the alkali metal carbonate(s) is required, and is followed by the MTM® or Mn Greensand, followed by either one or all of the other mentioned resins.

The amounts and proportions of the foregoing media are dependent on the amount of contaminated water, the flow rates, the amount of each contaminant, and the water pH. The amount necessary of each medium for the reduction or removal of each particular radionuclide can be determined by following the guidelines described by Dr. Robert C. Moore of Sandia National Laboratories. The ratio of alkali earth metal carbonate to MTM® or Mn Greensand ratio ranges from 1:3 to 1:10. The ratio of MTM®/Mn Greensand media required for radionuclides ranges from 1:10 to 1:1.

Depending on what is actually in the water; one or several elements/steps this filter system/method can be eliminated.

Depending on the contaminant(s) collected, it is understood that proper waste disposal of the media canisters, cartridges, housing, etc., will require compliance with the applicable local and federal governing regulations.

Removing or Reducing Toxic Heavy Metals and Other Hazardous Compounds from Water

In contrast to the prior art, the present invention provides the synergies of using combinations of filtration media and sediment filters to oxidize (if necessary), react with, adsorb, absorb or physically filter these heavy metals, such as As, Pb, and Cr and other compounds such as cyanides from drinking, washing and bath water.

Specifically, use of an embodiment of the Metsorb™ system as described herein, followed by MgO or Mg(OH)₂ proportioned in a 1:1 ratio provides synergistic filtering performance when compared to using either medium alone.

Also, CaCO₃, MgCO₃ and/or Li₂CO₃followed by MTM® or Mn Greensand are used if the particular heavy metal is found in its lower valence state in water insoluble form.

For example, in the case of trivalent vs. hexavalent chromium (Cr⁺³ vs. Cr⁺⁶), when using ion exchange resins technologies, Cr⁺⁶ is much easier to remove than Cr⁺³ . Therefore, pre-treating with the alkali earth metal followed by the MnO₂-coated medium would be employed before the use of the TiO₂ followed by the MnO₂. Also post filtering with, for example, a 0.1 to 10 μm sediment filter may be used to prevent subsequent bleeding of the filtration medium or heavy metal ions.

### Prevention or Diminution of Dermatitis by Reducing Iron, Bacteria and Toxins in Water

The present invention is also directed to a water filtration device, system and method that reduces dermatitis in a subject who uses water contaminated by C. perfringens bacteria and their toxins in his home or other site of water use. The present invention reduces, or, preferably, eliminates the bacteria and their toxins in or from the water supply.

In this embodiment, a mechanical pre-filter is used as a first step to remove silt followed by a filtration device/step devoted to insuring that the pH remains >7 by passing the water over crystalline CaCO₃, MgCO₃ and/or Li₂CO₃. This is followed by a filtration device/step that employs a MnO₂-coated medium, preferably zeolite or sand that oxidizes the bacteria, the toxins and any ferrous iron. The Mn oxide-coated medium traps the iron as well as any bacteria until they are destroyed. The next filtration element/step comprises activated carbon (granular or powdered, preferably granular which traps any toxin and bacterial fragments that pass through the previous section. This is preferably followed by a size exclusion filter with a pore size of between about 0.1 and about 40 μm, more preferably between about 0.1 and about 10 μm, to remove any activated carbon or MnO₂-coated medium fines. Because bacteria can grow in activated carbon, particularly when there are long periods of no fluid flow, the activated carbon is preferably treated with silver ions to prevent such growth.

It is further useful to interpose a step that provides peroxide to the water before treatment with the Mn-coated medium. This adds additional oxidizing capacity and provides a more aggressive oxidation potential to the water. Preferred are Ca- or Mg-peroxide in the form of tablets, pellets or powder. The release of the peroxide is preferably controlled, for example, by containment in a water permeable container, such as a porous ceramic, a plastic, fiber bag, or any other container that can hold the peroxide materials for controlled release, but still allow the water to pass at desired flow rates. These are known in the art.

Though MnO₂ is nearly insoluble, it nevertheless may serve as a source of Mn ions for bacteria that can use them in place of iron for RNR enzymatic activity.

Hence, combining the filtration media as described, creates a synergistic filtration system that greatly minimizes the risk of dermatitis for a person using the treated water for washing or bathing.

Having now generally described the invention, the same will be more readily understood through reference to the following examples which are provided by way of illustration, and are not intended to be limiting of the present invention, unless specified.

### EXAMPLES

### Example I

### Improved Products for Multiple Uses

The present invention includes an improvement in MetalTrap™ in which the CC:MTM® ratio in MetalTrap™ was lowered from 1:9 to 1:3 to increase the pH more effectively for both Fe and Cu and H₂S removal in well-water applications where influent water has a pH<6.8. In a preferred embodiment, the CC is replaced by magnesium carbonate (MgCO₃) (also abbreviated “MC”) in a 1:5 ratio with MTM®.

The present invention includes an improvement in the PureStart® filter device in which granular activated grades have been modified from HydroDarco 4000® to HydroDarco 3000® which is a larger granular with higher surface area and porosity making it more efficient.

The present invention includes a 3-part MetalTrap Ultra™ (MTU™) filter system which incorporates CC followed by MTM® followed by HD4000® in a ratio of 1:5:5. In a preferred embodiment of the foregoing, the ratios in the 3-part MTU™ filter system is 1:4:4 CC:MTM®:HD3000®.

In another embodiment preferably for aquarium use, a composition of 0.3% silver ions blended in granular activated carbon is utilized to prevent or minimize bacterial growth in the PureStart™ Bio filter system which uses HD4000®.

The present invention also provides a 5-part water filter system to remove or reduce radionuclides from contaminated water (of the type that resulted from the escaped radioactive gases emitted by the nuclear power plant in the 2011 earthquake in Japan). This system comprises a synergistic media in an equal volume basis of the following materials from inlet to outlet:
(1) Granular activated carbon (HD3000®); for radioactive Iodine (I) removal
(2) Silica Sand for radioactive plutonium (Pu) removal;
(3) Titanium dioxide for uranium-235 (²³⁵U) reduction;
(4) Ion exchange resin for reduction of radioactive cesium (Cs);
(5) Calcium Hydroxy Phosphate (Calcium Apatite) for additional removal of all radionuclides.

In another embodiment, the present invention provides an arsenic (As) removal filter system with advantages over those cited in the prior art above, based on a 4-stage synergistic system consisting of: 1 part MC:5 parts MTM®:5 parts titanium dioxide (TiO₂):5 parts magnesium oxide (MgO).

### Example 2

### Four Stage Water Filtration System

A 4-stage water filter system is used in a location where the levels of chlorine, heavy metals and organic contaminants are high. One such location is in the residential sections in and around Hong Kong, China. This system is for point-of-use applications for drinking water and employs: 1 part magnesium carbonate (MC):5 parts MTM®:5 parts granular activated carbon (GAC):3 parts TiO₂.

### Example 3

### Five Stage Water Filtration System

A 5 stage water filter system is used for a shrimp breeding farm in the Florida Keys where the source of water is unique. The water contains as high as 1 to 3 ppm chlorine, and high levels of iron, H₂S and heavy metals. The water is has high levels of contamination with bacteria that are detrimental to shrimp larvae. This filtration system effectively removes the above contaminants to provide a healthy environment for breeding shrimp. The filtration system employs 1 part CC:1 part MTM®:1 part GAC:1 part quaternary ammonium surface-coated zeolite medium:1 part polyester spun-wound filter of with average pore sizes of 1 μm.

### Example 5

### Treatment of Water for Bathing or Washing

As noted, above, disinfection of drinking water to remove coliform bacteria does not eliminate C. perfringens bacteria or their toxins. The oxidizing conditions at treatment plants promote dormancy of C. perfringens bacteria which are reactivated in the distribution system due to organic material that quickly makes the water reducing. This is a particular problem with water from sources in lowland regions, e.g., the Florida Keys and Miami, where the water never becomes oxidizing enough to remove the organic material. Bathing, showering or washing with this water will inoculate skin with these bacteria leading, as noted, to skin roughness and inflammation (dermatitis).

To eliminate both the C. perfringens and the toxins, the following filter system is employed. A mechanical pre filter that removes silt is followed by a section that insuring that the pH is above 7 by passing the water over crystalline CaCO₃, MgCO₃, Li₂CO₃ or a mixture thereof. This is followed by a section that oxidizes the bacteria, their toxins and any ferrous iron. This latter section utilizes MgO₂-coated Zeolite to trap any bacteria (which are destroyed) and the iron. The following section, comprising activated carbon treated with silver, traps any toxin and bacterial fragments that passed through the previous section. The silver prevents bacterial growth or any intact organisms that reach this stage. This section is followed by a 1 μm filter to contain any activated carbon or Zeolite fines.

A step of calcium peroxide treatment before the Mn-coated Zeolite adds additional oxidizing capacity and provide a more aggressive oxidation potential to the water. This is accomplished with calcium peroxide tablets or powder. The release or the calcium peroxide is controlled by use of a porous ceramic container, a plastic container with limited porosity or a ceramic fiber bag.

The references cited above are all incorporated by reference herein, whether specifically incorporated or not.

Having now fully described this invention, it will be appreciated by those skilled in the art that the same can be performed within a wide range of equivalent parameters, concentrations, and conditions without departing from the spirit and scope of the invention and without undue experimentation.

What is claimed is:

1. A liquid filtration system for removing or reducing the level of a contaminant from a liquid comprising the following filtration devices or elements serially disposed:
(a) an optional mechanical pre-filter;
(b) a bed of crystalline alkaline earth carbonate;
(c) an optional oxidation chamber that releases calcium peroxide, magnesium peroxide or sodium percarbonate into the liquid;
(d) a bed of MnO₂-coated medium;
(e) a bed of activated carbon optionally comprising silver ions at levels ranging from 0.001% to 3.0% (w/w); and
(f) an optional size exclusion filter.

2. The system of claim 1 wherein the liquid is water.

3. The system of claim 1 wherein the contaminant being removed is an inorganic metal or H₂S.

4. The system of claim 3 wherein the metal is Fe, Cu, Mn, Pb, As, Cr, Co, Ni, Al, Ag or Zn.

5. The system of claim 1 wherein the contaminant being removed is a radionuclide.

6. The system of claim 1 wherein the contaminant being removed is bacteria or a toxin produced by the bacteria.

7. A method for removing or reducing the level of a contaminant from a liquid comprising:
(a) filtering the liquid through an alkali metal carbonate to obtain a first filtrate;
(b) treating the first filtrate with a filtration medium coated with manganese dioxide.

8. The method of claim 7 further comprising a step of:
(c) filtering the liquid through a reducing or oxidizing ion exchange resin.

9. The method of claim 7 further comprising, before step (b), a step of
(c) treating the liquid with a surface-treated titanium dioxide.

10. The method of claim 9 further comprising after step (b) a step of:
(d) treating the liquid with granular and/or powdered activated carbon.

11. The method of claim 10 wherein the activated carbon is granular and to which is added a composition comprising 0.001 to 3.0% (w/w) silver ions that prevent or reduce bacterial growth in the liquid.

12. The method of claim 7, further comprising after step (b) a step of:
(e) filtering the liquid through a filter having a pore size between about 0.1 μm and about 40 μm.

13. The method of claim 10, further comprising after step (b) or (d) a step of:
(e) filtering the liquid through a filter having a pore size between about 0.1 μm and about 40 μm.

14. The method of claim 7, comprising, prior to step (b), a step of treating the liquid with a powdered oxidizing agent.

15. The method of claim 14 wherein said oxidizing agent is calcium peroxide, magnesium peroxide or sodium percarbonate.

16. The method of claim 7 wherein the liquid is water.

17. The method of claim 16 wherein the water is in a municipal water supply or a well.

18. The method of claim 7 wherein the alkali metal carbonate is CaCO₃, MgCO₃, Li₂CO₃, or a mixture thereof.

17. The method of any of claim 7, wherein the MnO₂ is MTM®, a form of Manganese Greensand, or another MnO₂-coated medium base.

18. The method of claim 17 wherein the medium base is sand, zeolite, activated carbon, an alkali metal carbonate or an oxide.

19. The method of claim wherein the contaminant is an inorganic metal.

20. The method of claim 19 wherein the inorganic metal is Fe, Cu, Mn, Pb, As, Cr, Co, Ni, Al, Ag, Au, or Zn.

21. The method of claim 7 wherein the contaminant is a radionuclide.

22. The method of claim 21 wherein the radionuclide is U, Cs, Pu, Ra, Co or I.

23. The method of claim 7 wherein the contaminant is bacteria or a toxin produced by the bacteria.

24. The method of claim 23 wherein the bacteria are Clostridium perfringens

25. A method of preventing or ameliorating a skin condition, disorder or disease in a subject having or being susceptible to said condition, disorder or disease, comprising providing to said subject a water supply used by said subject for bathing or washing water that has been treated by the method of claim 7.

26. The method of claim 25, wherein the condition, disease or disorder is dermatitis, eczema or psoriasis.

27. A method of preventing or ameliorating a skin condition, disorder or disease in a subject having or being susceptible to said condition, disorder or disease, comprising
(i) treating a water supply used by the subject with the following filtration and treatment steps:
(a) filtering the water through mechanical pre filter that removes silt;
(b) filtering the water through crystalline CaCO₃, MgCO₃, Li₂CO₃ or a mixture thereof;
(c) treating the water by controlled release of an oxidizing agent selected from the group consisting of calcium peroxide, magnesium peroxide and sodium percarbonate dispensed from a ceramic fiber bag with limited porosity;
(d) filtering to water through MgO₂-coated zeolite;
(e) treating the water with activated carbon to which is added a composition comprising 0.001 to 3.0% (w/w) silver ions; and
filtering the water liquid through a filter having a pore size of about 1 μm.
(ii) providing said treated, filtered water to said subject for bathing or washing.
